(19) United States
(12) Patent Application Publication
(10) Pub. No.: US 2010/0010482 A1
(43) Pub. Date: Jan. 14, 2010

(54) Enhanced Photodynamic Therapy Treatment and Instrument

(75) Inventor: Wolfgang Neuberger, Labuan (MY)

Correspondence Address: BOLESH J. SKUTNIK PhD,JD, 515 Shaker Road, East Longrneadow, MA, 01028, US

(73) Assignee: CeramOptec Industries Inc.

(21) Appl. No.: 12/482,616

(22) Filed: Jun. 11, 2009

Related U.S. Application Data
(60) Provisional application No. 61/074,796, filed on Jun. 23, 2008.

(51) Int. Cl. A61B 18/20; A61M 31/00

(52) U.S. Cl. 606/10; 604/500

(57) ABSTRACT

A device and method for tumor destruction by photodynamic therapy (PDT) is highly effective in that both the tumor cells themselves and the vasculature feeding the tumor are attacked in a single treatment. Local and systemic methods are used for injecting the photosensitizer (PS) into the tumor as well as vasculature. This combination brings about cellular and vascular destruction more effectively. In the first step, an initial PS dosage is administered locally to the tumor and allowed to achieve near-optimal tumor penetration. A second PS dosage is then administered systemically at a predetermined time after the first dosage and allowed to penetrate the tumor's vascular system for a period of time sufficient to produce a relatively high or otherwise predetermined concentration of PS in the vasculature. A relatively low dosage (mg/kg of PS to body weight) of photosensitizer is used for systemic administration; one advantage of this relatively low dosage is that it may help to help reduce skin photosensitivity. The tumor is then irradiated with a wavelength suitable to activate the first PS, followed by irradiation of the tumor vasculature with a wavelength suitable to activate the second PS. One advantage of the currently preferred embodiment is that both the tumor cells, and the associated vasculature, call be damaged to effectively destroy the tumor in a single treatment. This is achieved using one or two, relatively low photosensitizer dosage, in comparison to prior art dosage. Destruction of the vasculature terminates or otherwise substantially reduces the supply of blood to the tumor, thereby causing the death of any remaining tumor cells and preventing tumor regrowth. The device irradiates the treatment site with more than one wavelength and provides the ability to monitor PS concentration and manually or automatically control the timing of irradiation to activate one or more PSs in sequence or nearly simultaneously.

Domestic Priority under 35 USC 199(e). This application claims the benefit of U.S. Provisional Application Ser. No. 61/074,796, entitled “Enhanced PhotoDynamic Therapy Treatment and Instrument”, by Wolfgang Neuberger filed Jun. 23, 2008, which is incorporated by reference herein.

### BACKGROUND OF THE INVENTION

1. Field of the Invention

The present invention relates to Photo Dynamic Therapy of diseased tissue, particularly cancerous tissue and tumors. In particular, the present invention relates to a device and a dual-mode PhotoDynamic Therapy having multiple administrations of photosensitizer(s) to diseased tissue prior to irradiation and directly destroying cancerous tissue as well as vasculature feeding the cancerous tissue.

2. Information Disclosure Statement

PhotoDynamic Therapy (PDT) is a method for treatment of a number of diseases and disorder, for example, inflammation and hyperproliferative diseases, such as cancer. Hyperproliferative disease includes the skin disease psoriasis, as well as arthritis, a chronic inflammatory disease of the joints. In PDT, a photosensitizer (PS) is applied locally or systemically to the organism, and, generally, accumulates in diseased (e.g. hyperproliferative) tissue to a greater extent than in normal tissue Differential PS accumulation is a highly desirable goal in developing methods for PDT, because it helps to protect normal cells and tissues from damage inflicted by irradiation during PDT. This is especially true of the skin, which can sustain additional damage by normal daylight or indoor artificial lighting. So far, all known PS, when activated, cause damage to both healthy and diseased cells in their proximity. Thus, the ability of PS to preferentially accumulate in diseased tissue versus normal tissue is an important element of a beneficial treatment by PDT. Ideally, only malignant cells should be destroyed during PDT. The unaltered function of the surrounding normal, healthy cells and the continued presence of intact connective tissue fibers is the basis for good functional and structural (and in many cases, where applicable, good cosmetic) results with PDT.

U.S. Pat. No. 4,957,481 to Gatenby describes single or multiple local administrations of a PS directly into a tumor mass for covering a larger tumor area. For multiple administrations, each administration is spatially separated, so that a specific volume of tissue is exposed to PS. This patent teaches near simultaneous, spatially-separated, multiple administrations to establish and maintain a desired level of PS across a large volume of diseased tissue to achieve effective PDT treatment. A single PS dose per area to be treated is intended with this method, and further the method does not contemplate targeting tumor vasculature.

Another method involving repeated administrations of PSs is described in U.S. Pat. No. 5,298,018 to Narciso, which describes the use of PhotoDynamic Therapy (PDT) as an adjunctive or stand alone procedure for the treatment of cardiovascular disease, In particular, Narciso's method is specifically directed to prevent restenosis by blocking access of growth factor to binding sites in smooth muscle cells. The method relies on a pharmacokinetic therapy, with or without light therapy, using physical or chemical interactions between the PS and muscle cells to block the binding sites independent of any light therapy. In this method, a PS is administered prior to the surgical or interventional procedure and then readministered after the procedure to replace the PS which is cleared or washed out from the cells over time, thus maintaining a PS concentration in a level sufficient to block the binding sites. This method does not address PDT treatments of cancerous tissue such as tumors.

U.S. Pat. No. 6,240,925 to McMillan, et al. discloses methods for treating tumors or other vascular regions without significantly affecting surrounding tissue. In this method, a tumor mass is irradiated, in the presence of a bioreductive agent or a cytotoxin, with light of a wavelength and parameters sufficient to preferentially heat blood vessels supplying the tumor, such as through absorption by hemoglobin. In one embodiment, the blood vessels are heated to the point of denaturation to prevent delivery of oxygen to the tumor mass. Alternatively, a chromophore or photoactive compound is administered to the site of irradiation, in addition to the bioreductive agent, to selectively absorb the radiation wavelength.

U.S. Patent App. No. 2002/0026945 to Gomer et al. describes the use of PDT to directly damage tumor cells and inhibit blood flow supply to a tumor (photodynamic therapy mediated oxidative stress). To combat the activation of gene expression due to tissue hypoxia, combination procedures are provided that include anti-angiogenic treatments to improve the effectiveness of PDT. This invention advocates the administration of an anti-angiogenic agent before and after standard PDT. It does not suggest multiple administrations of PS for targeting of tumor cells and vasculature.

U.S. Pat. No. 5,576,013 to Williams et al. discloses a method of PDT used to target abnormal aggregations of vascular tissue or neovascular tissue supplying lesions such as tumors. A PS is applied to the target tissue followed by irradiation to induce photothrombosis within target vascular tissue, forming blood clots within the blood vessels to restrict blood flow to the lesion. This method is limited to treatment of blood vessels to indirectly harm the target lesion; it provides no method for directly killing cells in the lesion.

U.S. Pat. No. 6,443,976 to Flower, et al describes methods for treating conditions associated with abnormal vasculature, such as tumors. The method generally consists of applying PDT to a lesion/tumor, followed by thermal photocoagulation of the blood vessels feeding the tumor. Application of radiation in the photocoagulation step is described as being sufficient to heat the vessel and cause at least partial occlusion. In certain cases the photocoagulation step is accomplished with the help of radiation-absorbing dyes. The radiation-absorbing dyes may also fluoresce and be used in angiographic imaging. This patent does not contemplate the use of PS that is activated by radiation in order to destroy target tissue by non-thermal mechanisms (e.g., singlet oxygen production), which would be advantageous as they do not pose the risks associated with thermal effects and have a limited range of action.

US patent application number 2004/0147501 by Dolmans et al., describes a PDT method for simultaneously targeting the vasculature and tumor cells. In this invention, fractionated multiple drug dose are administrated to the patient before a single light administration. PS is administrated at different time points prior to the light administration. This ensures complete destruction of the tumor mass and also the blood vessels supplying the tumor with oxygen and nutrition. However, as described herein, the PS needs to be activated at correct time interval to destroy the tumor first, followed by blood vessels; otherwise, there is a greater chance for destruction of blood vessels without destroying the main tumor body. Due to multiple fractionated photosensitizers, the drug dose used increases the chances of skin sensitivity. As presently taught, the administration of multiple drug doses/dosage needs to be carried out at right interval of time to achieve the end results.

The ability to induce both hypoxia via vasculature destruction and the direct destruction of the hyperproliferating cells in a single treatment vastly improves the speed and efficiency of the treatment, as well as greatly reducing the risk of tumor regrowth or metastasis. Accordingly, a single treatment that destroys a cancerous tissue through both mechanisms would be beneficial.

### OBJECTIVES AND BRIEF SUMMARY OF THE INVENTION

It is an objective of the present invention to provide a method of tumor destruction that requires relatively short treatment times and more thorough treatment in comparison to certain prior art methods.

It is another objective of the present invention to provide a method of tumor destruction that targets both tumor cells and the blood vessels supplying the tumor.

It is yet another objective of the currently preferred embodiments of the present invention to provide a PDT method that produces high concentrations of photosensitizer(s) (“PS”) in both tumor cells and tumor vasculature simultaneously or during a single treatment.

It is still another objective of the currently preferred embodiments of the present invention to provide a device that optimizes method of tumor destruction by providing the ability to detect PS concentration and thereby irradiate a treatment area at optimal times.

It is a further objective of the currently preferred embodiment of the present invention to use local and systemic methods to inject PS into the tumor and vasculature, respectively for complete destruction of the tumor and reduced skin photosensitivity in comparison to prior art methods.

Briefly stated, the present invention is directed to a device and method for tumor destruction by photodynamic therapy (“PDT”) that is highly effective in that both the tumor cells themselves and the vasculature feeding the tumor are attacked in a single treatment. Local and systemic methods are used for introducing the photosensitizer (“PS”) into the tumor as well as vasculature. Alternatively photosensitizer may also be administered orally. This combination can bring about cellular and vascular destruction more effectively. In the first step, an initial PS dosage is administered locally to the tumor and allowed to achieve near-optimal tumor penetration. A second PS dosage is then administered systemically at a predetermined time after the first dosage and allowed to penetrate the tumor's vascular system for a period of time sufficient to produce a relatively high or otherwise predetermined concentration of PS in the vasculature. A relatively low dosage (mg/kg of PS to body weight) of photosensitizer is used for systemic administration. One advantage of this relatively low dosage is that it may help to reduce the skin photosensitivity. The tumor is then irradiated with a wavelength suitable to activate the first PS, followed by irradiation of the tumor vasculature with a wavelength suitable to activate the second PS. One advantage of the currently preferred embodiment is that both the tumor cells, and the associated vasculature, can be damaged to effectively destroy the tumor in a single treatment. This is achieved using one or two, relatively low photosensitizer dosage, in comparison to prior art dosage. Destruction of the vasculature terminates or otherwise substantially reduces the supply of blood to the tumor, thereby causing the death of any remaining tumor cells and preventing tumor regrowth. The device can irradiate the treatment site with more than one wavelength and provide the ability to monitor PS concentration and manually or automatically control the timing of irradiation to activate one or more PSs in sequence or nearly simultaneously.

The above and other objects, features and advantages of the present invention and of the currently preferred embodiment thereof will become apparent from the following description read in conjunction with the accompanying drawings.

### BRIEF DESCRIPTION OF FIGURES

FIG. 1: illustrates the concentration of the photosensitizer in the plasma over a period of time or time interval.

FIG. 2: illustrates an exemplary embodiment of the PDT treatment device of the invention.

### DETAILED DESCRIPTION OF PREFERRED EMBODIMENTS

The method of PDT described herein is very effective and is an improvement over prior art methods especially for cancerous or hyperproliferative tissues. Like other cells in the body, cancerous cells require oxygen and nutrients in order to continue to grow and multiply. A steady supply of oxygen and nutrients must be supplied through the vascular system to the cells in order for the hyperproliferative tissue to be sustained and to grow. The currently preferred embodiments of the present invention take advantage of this by attacking cancerous (hyperproliferative) tissue on two fronts with a dual-mode treatment. The first mode involves direct destruction of diseased tissue by activating photosensitizers (PS) accumulated in the tumorous tissue. The second mode indirectly attacks the diseased tissue by sufficiently damaging and/or closing a blood vessel that feeds the tumor tissue to prevent or substantially prevent the flow of blood to the target tumor tissue. As with traditional PDT treatments, the PS is activated by radiation to have a direct cytotoxic effect on targeted hyperproliferative cells. One advantage of the currently preferred embodiment of the present invention is that they use PS not only to destroy tumor cells, but also to destroy the tumor's vascular system preferably in a single treatment using two photosensitizers activated at different wavelength and more preferably, using a single photosensitizer in different dosage and by varying Drug-Light-Interval (DLI); i.e., the period of time between the administration of a photosensitizer agent and irradiation with energy.

### Definitions

1) The term “dosage” refers to drug dosage i.e., mg/kg of PS to body weight. The term “photosensitizer” mean photosensitizing agent which can be activated at a given wavelength. The photosensitizer of present invention can be selected from the group of but not limited to di-hydro and tetra-hydro porphyrins, cholrins and pthalocynines. The photosensitizer is suitably formulated for local and systemic administration. Alternatively oral administration can also be used, (U.S. Provisional Patent Application Ser. No. 61/173,487 filed Apr. 28, 2009, entitled “New Oral Formulation for Tetrapyrrole Derivates,” and U.S. Provisional patent Application Ser. No. 61/173,477 filed Apr. 28, 2009, entitled “Novel Photosensitizer Formulations for Oral Administration,” and corresponding non-provisional applications, which describe oral formulations of photosensitizer for oral administration, that may be used with present invention.).

2) This includes liposomal formulated photosensitizer, pegylated-Liposomal formulated photosensitizer or using suitable carrier system general known in the art.

3) The term “Low” dosage of photosensitizer is used in reference to the dosage that is used for administration to vasculature when compared to tumor cell.

As mentioned, PDT treatments generally focus on one of two targets; tumor tissue or the vasculature feeding the tumor. As such, one aim of PDT is the direct destruction of cancerous cells within a tumor. Some PS, such as meta-tetrahydroxyphenyl chlorin (mTHPC), known as Foscan™, are well suited for such treatments in that they preferentially accumulate in hyperproliferative tissue. Additionally, by targeting the tumor blood supply, the oxygen supply to the tumor cells can be cut off, resulting in tumor cell hypoxia. An example of a PS suitable for this approach is Pd-Bacteriopheophorbide (known as TOOKAD®, Steba Biotech N.V., Netherlands), which preferentially accumulates in the blood and thus is very useful for cancer treatments that attempt to destroy tumor vasculature.

The tumor vasculature destruction preferably is initiated after complete or substantially complete destruction of a target tumor mass. One advantage to this approach is that it may ensure sufficient oxygen supply for photo-cytotoxic effect in the tumor mass completely. This double effect of direct cell destruction and vasculature destruction ensures a more effective and more complete eradication of the diseased tissue in comparison to prior art methods.

Certain state of the art PDT treatments are lacking in that they fail to provide means and a method for producing a relatively high concentration of PS in both the tumor and the vasculature supplying the tumor, for activation during a single treatment. Typical treatments involve irradiation after a period of time sufficient to allow the PS to substantially clear out of healthy tissues while retaining an effective concentration in hyperproliferative tissue. Because PSs clear out of hyperproliferative (cancerous) tissue at a slower rate than healthy tissue, a period of time is determined, i.e., the Drug-Light-Interval (“DLI”), wherein an effective amount of PS remains in the tumor (diseased tissue) which is a significantly higher than that in healthy tissue. However, that period, (“standard DLI”) is generally too long to retain an effective concentration of PS in the vasculature supplying the tumor. Conversely, the period of time in which an effective concentration of PS remains in the vasculature is too short to allow an effective amount of PS to concentrate within the tumor. The dual-administration PDT method described herein provides a solution to this problem.

In a preferred embodiment, separate administrations of two distinct PS are performed prior to irradiation. In a first administration, a first PS is administered, locally or systemically, to the tumor mass. The administration may be local application or local injection, such as by injecting the first PS directly into the tissue mass or by intravenous injection. After administration, a certain period of time, (i.e., a first DLI₁), is allowed to pass in order for the molecules of the first PS to preferentially accumulate in the hyperproliferating cells. After a sufficient period of time (first DLI₁), near-optimal deep tissue concentration of the first PS is reached. The period of time required to achieve this concentration varies by PS and tissue type, but the time periods needed are readily determined by pre-testing or are known by those skilled in the art from previous treatments.

After sufficient near-optimal concentration of the first PS is reached, a low dosage of a second PS, having a different absorption wavelength than the first photosensitizer, is administered to the vasculature of the tumor. This may be accomplished, for example, through systemic intravenous injection or local intravenous injection. A sufficient amount of time (second DLI₂) is allowed to pass until optimal vascular cell concentration of the second PS is reached in the cells of the vessel walls.

The elapsed time period between administration of the second PS and irradiation of the second PS, i.e., DLI₂, varies between treatments, and can be determined by those skilled in the art, but is generally much shorter than the period of time required to produce an effective concentration of PS in the tumor tissue. For example, this second DLI₂ may be as short as few minutes to about 2 hours, and in other embodiments up to about 8 hours. In contrast, to standard PDT techniques the first DLI₁ period is often about, 24 to 48 hrs or up to, 24 to 96 hours or more, i.e., the elapses time between PS administration (systemic) and irradiation.

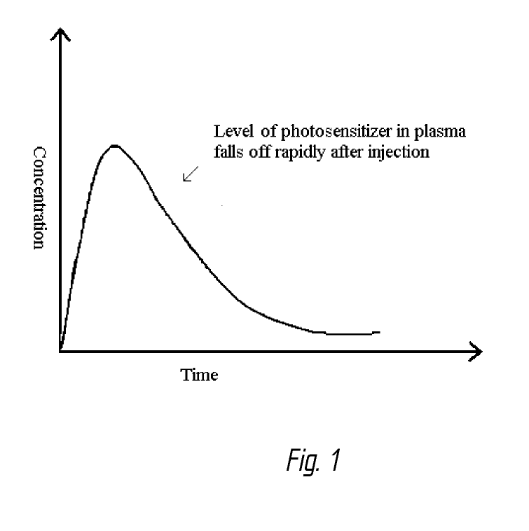

Several experimental studies have identified a discrepancy between times of maximal tumor drug uptake and optimum illumination intervals for the best tumor effect, both for temoporfin [meta-(tetrahydroxyphenyl) chlorin (mTHPC)] and for other PSs. Optimum illumination intervals are usually considerably shorter than times for maximal loading of the hyperproliferative tissue with sensitizer. It is thought that exposure of the endothelial cells of vessels feeding the tumor to the sensitizer determines tumor response, and that this is more closely reflected by plasma levels (see FIG. 1) than by tissue levels of PS. If a correlation between plasma drug levels and tumor response is a general phenomenon, then DLIs of a few hours could be considered for some clinical protocols. It has been previously reported in the art that PS such as meta-tetrahydroxyphenyl chlorin (mTHPC) accumulates in tumor vasculature much quicker than in tumor tissue, and that treatment protocols with a shortened drug-light-interval may be effective in treating the tumor (Cramers et al (2003)). The currently preferred embodiment of the present invention incorporates this principle in the described combination of the treatment that both effectively targets tumor tissue directly and targets tumor vasculature.

In another embodiment, the first and second PS are different and are each activatable by different wavelengths. The wavelengths can be delivered sequentially, preferably with minimal time elapsing between completion of tumor destruction and commencement of vasculature destruction. For example, a first irradiation may be applied to directly kill hyperproliferating cells making up the tumor, followed by a second irradiation to activate the second PS in the vascular regions to eliminate some or all of the tumor blood vessel function. The initial irradiation step destroys existing cancerous cells or greatly reduces them, and the latter irradiation step robs residual diseased tissue of its oxygen and nutrient supply to cause hypoxia of any remaining diseased cells as well as to prevent the growth of new tumor cells.

In another currently preferred embodiment, first PS is administrated either locally or systemically (depending on the location of the tumor mass). A second low dosage of PS is administrated intravenously/systemically, to allow the second PS to accumulate within the tumor vasculature. PS concentration is higher initially in the blood after systemic administration (see FIG. 1) hence the second PS will have a relatively short DLI as compared to first PS. Subsequent irradiation of the first PS kills some or all of the tumor cells, and irradiation of the second PS immediately or soon thereafter damages or occludes (partially or completely) the vasculature to quickly prevent further blood supply to the tumor. The second PDT action on the vasculature needs to be timed so that the oxygen supply for tumor is maintained to the extent necessary for the first photosensitizer to induce PDT in tumor cells.

In another currently preferred embodiment, first and second photosensitizers used are the same but with different DLI. The first and second PS may have same activation wavelength or a different activation wavelength to ensure complete destruction of tumor and blood vessel supplying the tumor cells.

Any number of PS and their derivatives may be used in conjunction with the present invention. A suitable PS must exhibit the characteristics of being non-toxic until activated by certain wavelengths, being cytotoxic upon photoactivation, and having a short range of influence. A preferred PS for use in the currently preferred embodiments of the present invention is meta-tetrahydroxyphenyl chlorin (m-TLIPC (temoporfin). This PS is particularly suitable for use in connection with the currently preferred embodiments of the present invention, because, although mTHPC is normally used to target tumor cells, its longer retention in hyperproliferating tissue makes it suitable for dual administration to treat both tumor cells and vasculature supplying blood. Other PS agents and their derivatives are described, for example, in U.S. Provisional Application Ser. No. 60/937/034, filed Jun. 22, 2007, entitled “Enhanced PhotoDynamic Therapy with Immune system Assist,” and corresponding non-provisional patent application Ser. No. 12/144,254 filed Jun. 23, 2008, which are both hereby incorporated by reference as part of the present disclosure in their entirety for all purposes.

It is generally desirable to destroy vasculature immediately after photodynamic treatment of the diseased tissue. This robs the tissue of blood—and thus oxygen and nutrients—as quickly as possible, in order to prevent any regeneration and to starve any remaining living diseased cells. Because the actual duration of radiation in PDT is usually very short compared to the drug-light interval (DLI), e.g., on the order of seconds or minutes, a peak concentration of each PS should be in place upon the first irradiation, so that the vasculature can be immediately irradiated after activation of the first PS, and reaction with tumor tissue, is complete.

This can be achieved by appropriately coordinating administration of the first PS (for tumor treatment) and the second PS (for treatment of vasculature). After the first PS is administered, a period of time sufficient for “near-optimal” tumor penetration, but less than that required for optimal tumor accumulation, elapses prior to administration of the second PS. The difference between these time periods is approximately the time required for optimal vasculature accumulation. Coordinating PS doses results in optimum concentrations of each PS in the tumor and vasculature, respectively, when irradiation is commenced.

In certain methods of the invention, the photosensitizing (PS) agent is administrated approximately simultaneously or contemporaneously with an effective amount of one or more immunostimulatory agents, which are known or become known to those skilled in the art. By way of nonlimiting example, immunostimulatory agents include TNF superfamily molecule such as CD40 ligand; agonists for TLRs; agonists for NAIP, CIITA, HET-E, TP-1-leucine-rich repeat pathway receptor, such as nucleotide-biding and oligomerization domain (NOD) 1, NOD2, and cryopyrin; chemokines; ILs; CSFs; IFNs; alarmins and purinergic P2X₇ receptor agonists. Especially effective are combination of agents which can elicit a massive expansion of antigen-specific CD8⁺ T cells and show unprecedented efficacy in vaccine and tumor models. For a discussion of a wide spectrum of immunostimulatory agents, see, Kombluth et al., J. Leukocyte boil., 80:1084-1102 (2006), which is incorporated herein by reference in its entirety. As one of skill in the art would recognize, the selection or amount of any particular immunostimulatory agent used depends on a number of factor such as potential for drug interaction; light sensitivity; patient age, height, and weight; potential side-effects; etc. which can be readily assessed on a case-by-case basis.

Photosensitizers and/or immunostimulatory agents as described herein are used in the disclosed methods in “effective amounts,” i.e., at a dosage that facilities the desired biological effects, for example blood vessel and/or tissue destruction, or immune system stimulation. An effective amount or dosage of a photosensitizer in the new methods depends, for example, on a variety of properties of the activating light (e.g., wavelength, energy, energy density, intensity), the optical properties of the target tissue, and properties of the photosensitizer. The upper and lower dosage limits depends on the type of photosensitizer used, and these limits are generally known and standardized by the manufacturers for a variety of photosensitizers. In addition the PS dosimetry can be determined empirically by those skilled in art utilizing well known methods, such as standard dose response measurements. Additional factors for consideration in determining the dosage per administration is the number of administration to be given, and tumor size or mass, and whether the dosage is to be administrated locally (i.e., at the diseased tissue site) or systemically. Thus, in fractional-PDT methods of the invention, the dosage can be lower than typically used with a given PS so that the total of all fractionated doses can be the same, lower, or higher than the standard dose for a given PS. As such, in certain embodiments, the effective dose utilized in the fractional-PDT methods of the invention range from about 0.1% to about 100% of the standard or manufacturer's recommended dosage.

Theoretically, the highest effective dose of the photosensitizer is limited by their toxicity to the subject, and the lowest effective dose is limited by the effectiveness of the photosensitizer for treating the disease at the low dose. For those skilled in the art, the example cited herein provides a methodology that will enable the photosensitizer dosimetry to be determined empirically. Exemplary doses contemplated by the present invention range from about 0.01 to about 10.0 mg/kg body weight(BW), and include, for example, 5.0, 2.5, 1.0, 0.5, 0.2, 0.25, 0.1, 0.09, 0.08, 0.07, 0.06, 0.05, 0.04, 0.03, or 0.02 mg/kg of BW. According depending on the type of PS used and the patient condition the dosage administrated would be variable. The dose per administration will depend on the total number of administration for a given total dose.

The elapsed time between irradiation of the tumor containing the first PS and irradiation of the vasculature containing the second PS should only be long enough to allow the phototoxic effect generated by the first irradiation to take effect. Because activation of the PS creates singlet oxygen which acts to destroy the proximate cancer cells, a sufficient amount of oxygen must be present in the tumor to allow this activation to take place. Destroying the vasculature too soon would cut off the oxygen supply to the tumor prematurely, reducing the effectiveness of the tumor destruction by halting the killing effect before the tumor is completely or substantially killed. This time period is variable, and depends on the amount of time required to achieve complete or substantial tumor destruction. Usually the second irradiation is given immediately after the completion of the first irradiation resulting in subsequent tumor and vasculature destruction.

In accordance with another currently preferred embodiment of the present invention, an irradiation device is provided that is well-suited to carrying out the above method. This irradiation device includes two main components: means for irradiating a treatment area with a plurality of wavelengths (e.g., at least two wavelength) and means for measuring or detecting the concentration of photosensitizer in tumor tissue and in vascular tissue. An additional feature of preferred embodiments of this device is an optional control means that triggers irradiation when a preselected concentration of the first and/or the second PS is reached.

The means for delivering activating radiation includes a treatment radiation source that includes one or more individual radiation sources that emit radiation substantially at a wavelength sufficient to activate each chosen PS (e.g. 652 nm for mTHPC). The treatment radiation source is capable of emitting numerous wavelengths, at least to activate each of the two chosen PS, which can be applied separately, preferably in a predetermined sequence. One currently preferred radiation source includes a diode source, such as a diode laser, an LED, a superluminescent diode, or a high power tapered diode such as a MOPA diode. The diode source may comprise numerous diodes, such as a diode bar or diode array. Other radiation sources, such as solid state lasers or non-coherent lamps may also be used. The capability of emitting numerous individual wavelengths can be accomplished by including two (or more) radiation emitters that emit selected wavelengths, such as numerous diodes or diode bars.

The treatment radiation source is coupled to a radiation delivery device by suitable coupling means such as a lens system. The radiation delivery device preferably consists of an optical fiber or optical fiber bundle. In one embodiment, the treatment radiation source is coupled to a plurality of optical fibers or optical fiber bundles for delivering radiation simultaneously or substantially simultaneously to a number of locations at or near the tumor. In another embodiment, the treatment radiation source may be coupled to a single optical fiber or fiber bundle, or numerous optical fibers or fiber bundles that are individually coupled to different individual radiation sources comprising the treatment radiation source. Other configurations can be selected to allow for sequential irradiation, and irradiation delivered to multiple locations.

Any of a variety means for detecting a concentration of PS in tissue that are currently known to those of ordinary skill in the pertinent art, or that later becomes known, may be employed in the device of the present invention. The detection means measures the PS concentration in the tumor cells as well as preferably in the epithelial cells. The detection means is preferably a device for measuring fluorescence of the PS to indicate PS concentration in tissue. An exemplary suitable device is a fluorescence-detecting probe inserted into tumor tissue or vascular tissue that detects PS fluorescence in vivo for a sample of tissue. The fluorescence-detecting probe may include a source of fluorescence-exciting radiation, which may preferably be the same wavelength as the PDT irradiation source, a fluorescence detector, means for delivering exciting radiation (wavelengths sufficient to cause PS fluorescence) to a treatment area, and means for receiving fluorescence emitted from the treatment area and delivering the fluorescence to a detector. Exciting radiation is radiation having preselected parameters (e.g. wavelength, power) sufficient to cause fluorescence that can be read by a detector to indicate a concentration of PS in the treatment area. The fluorescence detector may be, for example, one or more photodiodes.

In a preferred embodiment, a radiation delivery device with dual wavelength laser console and detection means is incorporated into a single unit connected to a control panel. The device is provided with an optical fiber for irradiating the treatment sites. Optical fibers for both PDT activating irradiation and fluorescence detection may be included in a single bundle that is directed to a treatment area or inserted directly into tissue. Furthermore, the same optical fibers (or other delivery devices) used for delivering activating radiation may also be used to both deliver fluorescence-exciting radiation and to return fluorescence from the treatment area to the fluorescence detector.

A control means, such as a computer or simple electronics incorporated into the delivery means, receives concentration information from the detection means. The control means may also include a display to indicate the concentration to a user. Additional input means, such as a keypad, may also be incorporated into the control means so that a user can set the desired concentration. In a preferred embodiment, the control means activates the treatment radiation source, and thus initiates activation of the PS, when a preselected concentration of a PS in the vasculature and/or the tumor tissue is reached. The control means can be set up to apply radiation of at least two wavelengths (one for activation of each of the two PS) in different sequences or patterns depending on the desired treatment.

The device described above is used in conjunction with the method described here to determine the time periods for ideal uptake of the PS in tumor tissue and vasculature. In a currently preferred embodiment of the present invention, a formulation containing mTHPC is intravenously injected into a patient having a cancerous tumor. The concentration of mTHPC in both normal tissue and tumor tissue is periodically measured until a maximum tumor concentration and minimum normal tissue concentration occurs. Thus, a desired time period in which ideal tumor selectivity is reached may be determined. This time may vary, and will likely be between 24 to about 48 hrs or about 24 to about 96 hours or 24 and about 110 hours, more likely between 90 and about 110 hours when systemically administered. In case of liposomally formulated PS drug light interval to about ˜4-12 hours. Similarly, time period for maximum vascular concentration after systemic administration can be a few minutes to about 2 hours, or in other embodiment up to about 8 hours.

The present invention is further illustrated by the following examples, but is not limited thereby.

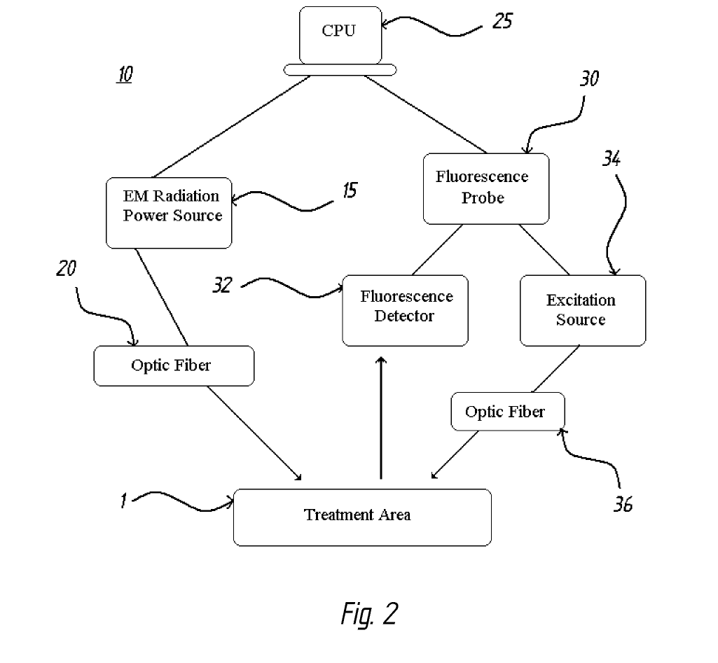

FIG. 2 depicts an exemplary PDT device 10 as presently described and claimed. The device comprises a means for providing radiation, for example, a Treatment Radiation Source FIG. 2 comprises an Electromagnetic (EM) radiation power source 15. In any of the embodiments described herein, the Treatment Radiation Source is capable of emitting a wavelength sufficient to activate at least one photosensitizer. In certain preferred embodiments, the Treatment Radiation Source, for example, the EM radiation Power source 15, is capable of emitting a wavelength sufficient to activate a first photosensitizer and a wavelength sufficient to activate a second photosensitizer.

Any number of radiation source can be used in the device of the invention, including a diode source, such as a diode laser, an LED, a superluminescent diode, or a high power tapered diode, such as a MOPA diode. The diode source may comprise numerous diodes, such as a diode bar or diode array. Other radiation sources, such as solid state laser or non-coherent lamps also may be used. The capability of emitting numerous individual wavelengths can be accomplished by including two (or more) radiation emitters that emit selected wavelength, such as numerous diodes or diode bars.

The EM radiation power source 15 is connected to radiation Delivery Means, for example, at least one optic Fiber 20 for delivery of radiation (e.g.) to the treatment area 1 (see FIG. 2). The treatment radiation source is coupled to a radiation delivery device by suitable coupling means such as a lens system. The radiation delivery device preferably comprises an optical fiber bundle 20. In one embodiment, the treatment radiation source is coupled to a plurality of optical fibers or optical fiber bundles for delivering radiation simultaneously or substantially simultaneously to a number of locations at or near the tumor. In another embodiment, the treatment radiation source may be coupled to a single optical fiber or fiber bundle, or numerous optical fibers or fiber bundles that are individually coupled to different individual radiation sources comprising the treatment radiation source.

The device of the invention can also comprise one or more Detection Means. For example, FIG. 2 illustrates an exemplary embodiment in which the Detection Means comprises at least one fluorescence probe 30. The fluorescence probe 30 may be disposed at a single treatment site or multiple probes can be located at two or more locations. The fluorescence probe 30 may further comprise a fluorescence detector 32, an excitation source 34, and at least one optic fiber 36 for delivery of excitation radiation, for example, Ultraviolet radiation (e.g.,). In certain embodiments, the optic fiber 36 or bundle of optic fibers is also used for detection and measurement of light emitted from the photosensitizer(s).

Various light source may be used as excitation source, including lasers, photodiodes, and lamps; xenon arcs and mercury vapor lamps in particular. A laser only emits light of high irradiance at a very narrow wavelength interval, typically under 0.01 nm, which makes an excitation monochromator or filter unnecessary. The disadvantage of this method is that the wavelength of a laser cannot be changed by much. A mercury vapor lamp is a line lamp, meaning it emits light near peak wavelength. By contrast, a xenon arc has a continuous emission spectrum with nearly constant intensity in the range from 300-800 nm and a sufficient irradiance for measurement down to just above 200 nm.

Filters and/or monochromators may be used in fluorimeters. A monochromator transmits light of an adjustable wavelength with an adjustable tolerance. The most common type of monochromators utilizes diffraction grating, that is, collimated light enters a grating and exits with a different angle depending on the wavelength. The monochromator can then select which wavelength to transmit. For allowing anisotropy measurements the addition of two polarization filters are necessary: One after the excitation monochromator or filter, and one before the emission monochromator or filter.

The fluorescence is most often measured at a 90° angle relative to the excitation light. This geometry is used instead of placing the sensor at the line of the excitation light at a 180° angle in order to avoid interference of the transmitted excitation light. No monochromator is perfect and it will transmit some stray light, that is, light with other wavelength that the targeted. An ideal monochromator would only transmit light in the specific range and have a high wavelength-independent transmission. When measuring at a 90° angle, only the light scattered by the sample causes stray light. This results in a better signal-to-noise ratio, and lowers the detection limit by approximately a factor 10000, when compared to the 1800 geometry. Furthermore, the fluorescence can also be measured from the front, which is often done for turbid samples.

The detector can either be single-channeled or multichanneled. The single channeled detector can only detect the intensity of one wavelength at a time, while the multichanneled detects the intensity at all wavelengths simultaneously, making the emission monochromator or filter unnecessary. The different types of detector have both advantages and disadvantages.

The most versatile fluorimeters with dual monochromators and a continuous excitation light source can record both an excitation spectrum and a fluorescence spectrum. When measuring florescence spectra, the wavelength of the excitation light is kept constant, preferably at a wavelength of high absorption, and the emission monochromator scans the spectrum. For measuring excitation spectra, the wavelength passing thought the emission filter or monochromator is kept constant and the excitation monochromator is scanning. The excitation spectrum generally is identical to the absorption spectrum as the florescence intensity is proportional to the absorption.

Finally, the device of the invention may also include a Control Means connected to the Detection Means, and the Treatment Radiation Source. For example, FIG. 2 illustrates an exemplary embodiment in which the Control Means is a computer 25 (i.e., “CPU”). The computer receives information from the fluorescence probe 30, which indicates the concentration of photosensitizer(s) in the treatment area 1. The computer 25 is capable of triggering activation of the EM Radiation Source when a preselected, i.e., v\user defined, concentration of at least one of a first photosensitizer and a second photosensitizer are detected.

Additional configurations and operation modes are contemplated by the present invention, and will be evident to those of skill in the art. As such, the configuration illustrated in FIG. 2 is exemplary, and is not to be construed as limiting on scope of the invention.

PDT Using Two Different PS having Different Activation Wavelength

### Example 1

An exemplary treatment is described below utilizing the PS m-THPC (Foscan™) for tumor destruction and Pd-Bacteriopheophorbide (TOOKAD) for vasculature destruction. If preferred time periods for ideal uptake of the PS in tumor tissue and vasculature are known, treatment may commence as described below.

After the period is determined, a formulation containing Pd-Bacteriopheophorbide is intravenously injected and the concentration of Pd-Bacteriopheophorbide in tumor vasculature is periodically measured to determine the ideal vascular concentration.

Based on this data, administrations of mTHPC and Pd-Bacteriopheophorbide are coordinated so that ideal selectivity is reached in both the tumor and vasculature at substantially the same time. The device of the present invention may be used to automatically apply each PS and/or irradiation based on known parameters.

The tumor is then irradiated with a wavelength of about 652 nm, preferably with an irradiance of about 100 mW/cm². The irradiation time will vary, but should be sufficient to supply 20 J/cm² of energy to the tumor.

After a period of time sufficient to allow for maximum tumor tissue destruction, the tumor vasculature is irradiated with a wavelength around 762 nm. As above, the irradiance is preferably about 100 mW/cm², for a sufficient time to supply about 20 J/cm² of energy to the vasculature to achieve closure of the blood vessels.

### Example 2

Another exemplary treatment is described below utilizing the PS, mTHPC (Foscan™) for tumor destruction and mTHPBC (510,15,20-tetrakis(m-hydroxyphenyl)chlorin) (SQN400) for vasculature destruction. If the treatment periods are known the treatment could be administered to the tumor and vasculature. If the time periods are not known the following method may be used to determine the time periods.

The device described above is used in conjunction with the method described above. A formulation containing mTHPC [(˜0.8 mg/ml m-THPC) with propylene glycol and pure ethanol (˜3:2 v/v) as excipient] is injected intravenously into a patient having a cancerous tumor. The concentration of mTHPC in both normal tissue and tumor tissue is periodically measured until a maximum tumor concentration and minimum normal tissue concentration is achieved. Thus, a desired time period in which ideal tumor selectivity is reached may be determined. This time may vary, and will likely be less than 12 hours, more likely between 1 and about 5 hours.

After this period is determined, a formulation containing mTHPBC is injected intravenously and the concentration of mTHPBC in tumor vasculature is periodically measured to determine the ideal vascular concentration.

Based on this data, administrations of mTHPC and mTHPBC are coordinated so that ideal selectivity is reached in both the tumor and vasculature at substantially the same time. The device of the present invention may be used to automatically apply each PS and/or irradiation based on known parameters.

The tumor is then irradiated with a first wavelength of about 652 nm, preferably with an irradiance of about 100 mW/cm². The irradiation time will vary, but should be sufficient to supply 20 J/cm² of energy to the tumor.

After a period of time sufficient to allow for maximum tumor tissue destruction, the tumor vasculature is irradiated with a second wavelength of about 740 nm. As above, the irradiance is preferably about 100 mW/cm², for sufficient time to supply about 10 J/cm² of energy to the vasculature to achieve closure of the blood vessels.

PDT Method Using Same PS with Different Drug Light Intervals

### Example 3

In another example a single photosensitizer, mTHPC, is employed for destruction of both tumor and the vasculature by effectively modifying the Drug Light Interval (DLI). Accordingly, the first dosage of mTHPC is administered and is allowed to accumulate in tumor mass, followed by, irradiation with a wavelength of 652 nm. This ensures tumor mass destruction. This is followed by second low dosage of mTHPC which is targeted at the blood vessels supplying oxygen and nutrition to the tumor mass. The DLI for the second dose is very short compared to that for tumor accumulation, as the required concentration of PS in blood is achieved immediately after intravenous drug administration. After the relatively short DLI, light is illuminated at the targeted region leading to local generation of cytotoxic reactive oxygen species which then causes rapid vascular occlusion and tumor necrosis.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims. U.S. Provisional Patent Application Serial No. 60.937,034 filed Jun. 22, 2007, entitled “Enhanced PhotoDynamic therapy with Immune System Assist,” and corresponding nonprovisional patent application Ser. No. 12/144,254 filed Jun. 23, 2008, which describes PDT composition and methods which may be used with present invention are hereby incorporated by reference as part of the present disclosure in their entirety for all purpose. According, the detailed description of the currently preferred embodiment is to be taken in an illustrative as opposed to a limiting sense.

What is claimed is:

1. A method for the photodynamic treatment of hyperproliferative tissue, comprising the steps of:
a. administering an effective amount of a first photosensitizer to the hyperproliferative tissue;
b. allowing a first period of time to elapse until a desired concentration of the first photosensitizer accumulates in said hyperproliferative tissue;
c. administering an effective amount of a second photosensitizer to vasculature, feeding said hyperproliferative tissue;
d. allowing a second period of time to elapse until a desired concentration of the second photosensitizer accumulates in the vasculature feeding the hyperproliferative tissue;
e. applying radiation having a first wavelength to said hyperproliferative tissue such that said first photosensitizer is activated to destroy at least a portion of the hyperproliferative tissue; and
f. applying radiation having a second wavelength to said vasculature such that the second photosensitizer is activated to at least one of destroy at least a portion of and close said vasculature.

2. The method according to claim 1 wherein the first period of time and the second period of time are each of a length sufficient to produce substantially predetermined concentration of said first photosensitizer in the hyperproliferative tissue and a substantially predetermined concentration of said second photosensitizer in the vasculature with a small time delay between the first and second time periods.

3. The method according to claim 1, wherein the first and second photosensitizer and the said first and second wavelength are same.

4. The method according to claim 1, wherein the first and second photosensitizers and the said first and second wavelength are the same but exhibit different Drug-Light-Intervals.

5. The method according to claim 1, wherein said first photosensitizer is meta-tetrahydroxyphenylchlorin (mTHPC), and wherein said second photosensitizer is a low dosage of Pd-Bacteriopheophorbide

6. The method according to claim 1, wherein said first photosensitizer is meta-(tetrahydroxyphenyl)chlorin (mTHPC), and the second photosensitizer is 5,10,15,20-tetrakis(m-hydroxyphenyl) chlorin (mTHPBC).

7. The method according to claim 3, wherein said first photosensitizer is meta-tetrahydroxyphenylchlorin (mTHPC), and the second photosensitizer is a low dosage of mTHPC.

8. A device for the photodynamic treatment of hyperproliferative tissue useful for carrying out the method of claim 1, comprising:
i. a treatment radiation source capable of emitting a wavelength sufficient to activate a first photosensitizer and a wavelength sufficient to activate a second photosensitizer;
ii. at least one means to deliver radiation from said radiation source to a treatment area; and
means to detect a concentration of at least one of said first photosensitizer in hyperproliferative tissue and said second photosensitizer in vasculature feeding said hyperproliferative tissue.

9. The device according to claim 8, wherein said treatment radiation source is at least one radiation source.

10. The device according to claim 9, wherein said at least one radiation source is selected from a group consisting of a laser source, a non-coherent lamp, and a diode source.

11. The device according to claim 10, wherein said diode source is selected from the group consisting of a diode laser, a light-emitting diode, a superluminescent diode, and a tapered diode.

12. The device according to claim 8, wherein said at least one radiation delivery means comprises at least one waveguide selected from the group consisting of an optical fiber and an optical fiber bundle.

13. The device according to claim 10 and claim 12, wherein at least one radiation delivery means comprises at least one waveguide selected from the group consisting of an optical fiber and an optical fiber bundle comprising multiple optical fibers, and wherein individual optical fibers are optically connected to different radiation sources.

14. The device according to claim 8, wherein said detection means is a fluorescence-detecting probe, comprising:
a fluorescence-exciting radiation source;
a fluorescence detector;
means for delivering fluorescence-exciting radiation to a treatment area; and
means for receiving fluorescence from at least one of said first photosensitizer and said second photosensitizer and delivering said fluorescence to said fluorescence detector.

15. The device according to claim 14, wherein said fluorescence detector is at least one photodiode.

16. The device according to claim 14, wherein said means for delivering fluorescence-exciting radiation and said means for receiving fluorescence comprises at least one optical fiber.

17. The device according to claim 8, further comprising a control means connected to said detection means and said treatment radiation source, wherein said control means triggers activation of said treatment radiation source when a preselected concentration of at least one of said first photosensitizer and said second photosensitizer is detected.

18. The device according to claim 8, wherein said treatment radiation source comprises a first radiation source capable of emitting a first wavelength sufficient to activate said first photosensitizer, a second radiation source capable of emitting a second wavelength sufficient to activate said second photosensitizer.

19. The device according to claim 8, wherein said first radiation source and said second radiation source are activatable in a predetermined sequence.

20. The device according to claim 18, further comprising a control means connected to said detection means and said treatment radiation source, wherein said control means triggers activation of said treatment radiation sources in a predetermined sequence/and pattern when a preselected concentration of at least one of said first photosensitizer and said second photosensitizer is detected.

21. The device according to claim 17 or 20, wherein said control means is a computer.

22. The device for the photodynamic treatment of hyperproliferative tissue useful for carrying out the method of claim 1, comprising:
i. a treatment radiation source capable of emitting a wavelength sufficient to activate a first photosensitizer and a wavelength sufficient to activate a second photo sensitizer;
ii. at least one optic fiber for delivering radiation from said radiation source to a treatment area; and
iii. a fluorescence probe comprising an excitation source, and a fluorescence detector, wherein the excitation source delivers radiation to the hyperproliferative tissue through at lease one optic fiber, and wherein the probe monitors a concentration of at least one of said photosensitizer in hyperproliferative tissue and said second photosensitizer in vasculature feeding said hyperproliferative tissue.

23. The device of claim 22, further comprising a computer, wherein the computer is integrated with the treatment radiation source and the fluorescence probe and wherein the computer receives data from fluorescence probe and is capable of triggering activation of said treatment radiation source when a preselected concentration of at least one of said first photosensitizer and said second photosensitizer is detected.
